(19) United States
(12) Patent Application Publication
(10) Pub. No.: US 2024/0174734 A1
(43) Pub. Date: May 30, 2024

(54) PARVOVIRUS ANTIBODY

(71) Applicant: YntoAb Co., Ltd., Seongnam-si (KR)

(72) Inventors: Sung Young LEE, Guri-si (KR); Na Ra Shin, Gwangju-si (KR)

(73) Assignee: YntoAb Co., Ltd., Seongnam-si (KR)

(21) Appl. No.: 18/059,759

(22) Filed: Nov. 29, 2022

(30) Foreign Application Priority Data
Nov. 25, 2022 (KR) 10-2022-0159980

(51) Int. Cl. C07K 16/08; A61P 31/20

(52) U.S. Cl. CPC C07K 16/081; A61P 31/20; A61K 2039/505

(57) ABSTRACT

Antibodies that bind parvovirus are disclosed. The antibodies can be used in methods for preventing or treating parvovirus infection.

### CROSS REFERENCE TO RELATED APPLICATIONS

This application claims priority based on Korean Patent Application No. 10-2022-0159980 filed Nov. 25, 2022, the content of which is incorporated by reference in its entirety.

### INCORPORATION BY REFERENCE OF SEQUENCE LISTING

The content of the electronically submitted sequence listing, file name: Q282796_Sequence_Listing_As_Filed.txt; size: 41,817 bytes; and date of creation: Nov. 29, 2022, filed herewith, is incorporated herein by reference in its entirety.

### BACKGROUND

### 1. Field

Provided are relating to parvovirus antibodies, monoclonal parvovirus antibodies can be used in methods to prevent and/or treat parvoviral infection in subjects, such as dogs and cats. For example, the parvovirus antibodies provided may be used to provide passive immunity against infection with a canine or feline parvovirus.

### 2. Discussion of Related Art

Canine parvovirus type 2 (CPV) is a contagious virus occurring in dogs that has been thought to have originated from cats. Canine parvovirus (CPV) is the most important enteric virus infecting dogs worldwide. CPV virions are non-enveloped DNA viruses. There are multiple variants of the original CPV, including CPV-2a, CPV-2b and CPV-2c. The variants of CPV-2 differ from each other in only a few amino acids. It is assumed that the feline panleukopenia virus mutated to CPV2. Parvovirus is highly contagious and spreads directly or indirectly from dog to dog through feces and saliva. It is known that the mortality rate in untreated dogs in infected reaches about 91%.

Canine parvovirus is treated using hyperimmune serum. As the hybridoma fusion technology developed by Kohler and Milstein in 1975 and the production of humanized antibodies that humanize mouse monoclonal antibodies through genetic manipulation became possible, mass production of monoclonal antibodies became possible for therapeutic use. Since the foundation for the use of antibodies was laid, in humans, the technology of isolating B lymphocytes with pathogen-specific antibodies from patients infected with pathogens and cloning and expressing the monoclonal antibody genes has been utilized for the development of therapeutic antibodies.

Phage expression technology with avast human B cell antibody gene library of about 100 million or more makes it possible to easily and quickly select antibodies with high affinity. However, in the current veterinary field, antibodies for virus treatment are mainly produced using antisera of host animals or laboratory animals (rabbits, etc.), or monoclonal antibodies using mouse hybridomas are limitedly used in veterinary hospitals. However, it is not commercialized as an antibody treatment. In addition, the production of antibody therapeutics using animals has a long production period (6-12 months), animal welfare problems, and the possibility of contamination of infectious agents in antibody-producing donor animals.

### SUMMARY

The present invention is directed to Parvovirus monoclonal antibodies to reduce or eliminate the severity of morbidity and mortality associated with CPV.

The present invention is directed to Parvovirus monoclonal antibodies to prevent CPV infection. Objects of the present invention are not limited to those mentioned above, and other objects not mentioned above will be clearly understood by those skilled in the art from the description below.

Antibodies that bind canine parvovirus and/or feline parvovirus are provided. Antibody heavy chains and light chains that are capable of forming antibodies that bind canine parvovirus and/or feline parvovirus are also provided. In addition, antibodies, heavy chains, and light chains comprising one or more particular complementary determining regions (CDRs) are provided. Polynucleotides encoding antibodies to canine parvovirus and/or feline parvovirus are provided. Methods of producing or purifying antibodies to canine parvovirus and/or feline parvovirus are also provided. Methods of providing passive immunity against infection with a canine or feline parvovirus and/or treatment of parvoviral infection using antibodies to canine parvovirus and/or feline parvovirus are provided.

The following definitions of terms used herein are provided.

As used herein, numerical terms such as Kd are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

Novel antibodies directed against parvovirus are provided, for example antibodies that bind to canine parvovirus and/or feline parvovirus. Parvovirus antibodies provided herein include, but are not limited to, monoclonal antibodies, chimeric antibodies, caninized antibodies, and felinized antibodies.

Also provided herein are chimeric canine, chimeric feline, caninized, and felinized antibodies derived from Mab A, Mab B, Mab A v2, and Mab B v2. In some embodiments, amino acid sequences of caninized and felinized Mab A, Mab B, Mab A v2, and Mab B v2 are provided.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments (such as Fab, F(ab′)2, ScFv, minibody, diabody, triabody, and tetrabody) so long as they exhibit the desired antigen-binding activity. Canine, feline, and equine species have different varieties (classes) of antibodies that are shared by many mammalians.

The term antibody includes, but is not limited to, fragments that are capable of binding to an antigen, such as Fv, single-chain Fv (scFv), Fab, Fab′, di-scFv, sdAb (single domain antibody) and (Fab′)2 (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen combining sites and is still capable of cross-linking antigen. The term antibody also includes, but is not limited to, chimeric antibodies, humanized antibodies, and antibodies of various species such as mouse, human, cynomolgus monkey, canine, feline, equine, etc. Furthermore, for all antibody constructs provided herein, variants having the sequences from other organisms are also contemplated. Thus, if a murine version of an antibody is disclosed, one of skill in the art will appreciate how to transform the murine sequence based antibody into a cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, mouse scFv or a canine scFv). This denotes the sequences of at least part of the non-CDR regions, rather than the source of the construct. In some embodiments, the antibodies comprise a label or are conjugated to a second moiety.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, 3H, 14C, 35S, 90Y, 99Tc, 111In, 125I, 131I, 177Lu, 166Ho, or 153Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

The term “monoclonal antibody” refers to an antibody of a substantially homogeneous population of antibodies, that is, the individual antibodies comprising the population are identical except for possible natural-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

In some embodiments, the monoclonal antibody is Mab A, Mab A v2, Mab B, or Mab B v2.

“Amino acid sequence,” means a sequence of amino acids residues in a peptide or protein. The terms “polypeptide” and “protein” are used interchangeably to refer to a polymer of amino acid residues and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“Parvovirus” as used herein refers to any naturally occurring parvovirus or parvovirus variant, and includes canine parvovirus (CPV), such as CPV-2a, CPV-2b, and CPV-2c, and feline parvovirus (panleukopenia virus).

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three dimensional structural characteristics as well as specific charge characteristics. Epitopes can be formed both from contiguous or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “CDR” means a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, or a combination of the Kabat, Chothia, AbM, or contact definitions. The various CDRs within an antibody can be designated by their appropriate number and chain type, including, without limitation as CDR-H1, CDR-H2, CDR-H3, CDR-L1, CDR-L2, and CDR-L3. The term “CDR” is used herein to also encompass a “hypervariable region” or HVR, including hypervariable loops.

The term “variable region” as used herein refers to a region comprising at least three CDRs. In some embodiments, the variable region includes the three CDRs and at least one framework region (“FR”). The terms “heavy chain variable region” or “variable heavy chain” are used interchangeably to refer to a region comprising at least three heavy chain CDRs. The terms “light chain variable region” or “variable light chain” are used interchangeably to refer to a region comprising at least three light chain CDRs. In some embodiments, the variable heavy chain or variable light chain comprises at least one framework region. In some embodiments, an antibody comprises at least one heavy chain framework region selected from HC-FRT, HC-FR2, HC-FR3, and HC-FR4. In some embodiments, an antibody comprises at least one light chain framework region selected from LC-FR1, LC-FR2, LC-FR3, and LC-FR4. The framework regions may be juxtaposed between light chain CDRs or between heavy chain CDRs. For example, an antibody may comprise a variable heavy chain having the following structure: (HC-FR1)-(CDR-H1)-(HC-FR2)-(CDR-H2)-(HC-FR3)-(CDR-H3)-(HC-FR4). An antibody may comprise a variable heavy chain having the following structure: (CDR-H1)-(HC-FR2)-(CDR-H2)-(HC-FR3)-(CDR-H3). An antibody may also comprise a variable light chain having the following structure: (LC-FR1)-(CDR-L1)-(LC-FR2)-(CDR-L2)-(LC-FR3)-(CDR-L3)-(LC-FR4). An antibody may also comprise a variable light chain having the following structure: (CDR-L1)-(LC-FR2)-(CDR-L2)-(LC-FR3)-(CDR-L3).

The term “constant region” as used herein refers to a region comprising at least three constant domains. The terms “heavy chain constant region” or “constant heavy chain” are used interchangeably to refer to a region comprising at least three heavy chain constant domains, CH1, CH2, and CH3. Nonlimiting exemplary heavy chain constant regions include γ, δ, α, ε, and μ. Each heavy chain constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an al constant region) and IgA2 (comprising an α2 constant region) antibodies; and IgM antibodies include, but are not limited to IgM1 and IgM2. The terms “light chain constant region” or “constant light chain” are used interchangeably to refer to a region comprising a light chain constant domain, CL. Nonlimiting exemplary light chain constant regions include κ and λ. Non-function-altering deletions and alterations within the domains are encompassed within the scope of the term “constant region” unless designated otherwise. Canine and feline have antibody classes such as IgG, IgA, IgD, IgE, and IgM. Within the canine IgG antibody class are IgG-A, IgG-B, IgG-C, and IgG-D. Within the feline IgG antibody class are IgG1, IgG2a, and IgG2b.

The term “chimeric antibody” or “chimeric” refers to an antibody in which a portion of the heavy chain or light chain is derived from a particular source or species, while at least a part of the remainder of the heavy chain or light chain is derived from a different source or species. In some embodiments, a chimeric antibody refers to an antibody comprising at least one variable region from a first species (such as mouse, rat, cynomolgus monkey, etc.) and at least one constant region from a second species (such as human, dog, cat, equine, etc.). In some embodiments, a chimeric antibody comprises at least one mouse variable region and at least one canine constant region. In some embodiments, a chimeric antibody comprises at least one mouse variable region and at least one feline constant region. In some embodiments, all of the variable regions of a chimeric antibody are from a first species and all of the constant regions of the chimeric antibody are from a second species. In some embodiments, a chimeric antibody comprises a constant heavy chain region or constant light chain region from a companion animal. In some embodiments, a chimeric antibody comprises a mouse variable heavy and light chains and a companion animal constant heavy and light chains. For example, a chimeric antibody may comprise a mouse variable heavy and light chains and a canine constant heavy and light chains; a chimeric antibody may comprise a mouse variable heavy and light chains and a feline constant heavy and light chains; or a chimeric antibody may comprise a mouse variable heavy and light chains and an equine constant heavy and light chains.

A “canine chimeric,” “chimeric canine,” or “canine chimeric antibody” refers to a chimeric antibody having at least a portion of a heavy chain or a portion of a light chain derived from a dog. A “feline chimeric,” “chimeric feline,” or “feline chimeric antibody” refers to a chimeric antibody having at least a portion of a heavy chain or a portion of a light chain derived from a cat. In some embodiments, a canine chimeric antibody comprises a mouse or rat variable heavy and light chains and a canine constant heavy and light chains. In some embodiments, a feline chimeric antibody comprises a mouse or rat variable heavy and light chains and a feline constant heavy and light chains.

In some embodiments, a parvovirus antibody comprises a canine heavy chain constant region selected from an IgG-A, IgG-B, IgG-C, and IgG-D constant region.

In some embodiments, a parvovirus antibody comprises a feline heavy chain constant region selected from an IgG1, IgG2a, and IgG2b constant region.

A “caninized antibody” means an antibody in which at least one amino acid in a portion of a non-canine variable region has been replaced with the corresponding amino acid from a canine variable region. In some embodiments, a caninized antibody comprises at least one canine constant region (e.g., a γ constant region, an α constant region, a δ constant region, an ε constant region, a μ constant region, or etc.) or fragment thereof. In some embodiments, a caninized antibody is an antibody fragment, such as Fab, scFv, (Fab′)2, etc. The term “caninized” also denotes forms of non-canine (for example, murine) antibodies that are chimeric immunoglobulins, immunoglobulin chains, or fragments thereof (such as Fv, Fab, Fab′, F(ab′)2 or other antigen-binding sequences of antibodies) that contain minimal sequence of non-canine immunoglobulin. Caninized antibodies can include canine immunoglobulins (recipient antibody) in which residues from a CDR of the recipient are substituted by residues from a CDR of a non-canine species (donor antibody) such as mouse, rat, or rabbit having the desired specificity, affinity, and capacity. In some instances, Fv framework region (FR) residues of the canine immunoglobulin are replaced by corresponding non-canine residues. Furthermore, the caninized antibody can comprise residues that are found neither in the recipient antibody nor in the imported CDR or framework sequences, but are included to further refine and optimize antibody performance.

In some embodiments, at least one amino acid residue in a portion of a rat or a mouse variable heavy chain or a rat or a mouse variable light chain has been replaced with the corresponding amino acid from a canine variable region. In some embodiments, the modified chain is fused to a canine constant heavy chain or a canine constant light chain.

A “felinized antibody” means an antibody in which at least one amino acid in a portion of a non-feline variable region has been replaced with the corresponding amino acid from a feline variable region. In some embodiments, a felinized antibody comprises at least one feline constant region (e.g., a γ constant region, an α constant region, a δ constant region, an ε constant region, a μ constant region, or etc.) or fragment thereof. In some embodiments, a felinized antibody is an antibody fragment, such as Fab, scFv, (Fab′)2, etc. The term “felinized” also denotes forms of non-feline (for example, murine) antibodies that are chimeric immunoglobulins, immunoglobulin chains, or fragments thereof (such as Fv, Fab, Fab′, F(ab′)2 or other antigen-binding sequences of antibodies) that contain minimal sequence of non-feline immunoglobulin. Felinized antibodies can include feline immunoglobulins (recipient antibody) in which residues from a CDR of the recipient are substituted by residues from a CDR of a non-feline species (donor antibody) such as mouse, rat, or rabbit having the desired specificity, affinity, and capacity. In some instances, Fv framework region (FR) residues of the feline immunoglobulin are replaced by corresponding non-feline residues. Furthermore, the felinized antibody can comprise residues that are found neither in the recipient antibody nor in the imported CDR or framework sequences, but are included to further refine and optimize antibody performance.

In some embodiments, at least one amino acid residue in a portion of a mouse variable heavy chain or a mouse variable light chain has been replaced with the corresponding amino acid from a feline variable region. In some embodiments, the modified chain is fused to a feline constant heavy chain or a canine constant light chain.

A “fragment crystallizable polypeptide” or “Fe polypeptide” is the portion of an antibody molecule that interacts with effector molecules and cells. It comprises the C-terminal portions of the immunoglobulin heavy chains. As used herein, an Fc polypeptide includes fragments of the Fc domain having one or more biological activities of an entire Fc polypeptide. An “effector function” of the Fc polypeptide is an action or activity performed in whole or in part by any antibody in response to a stimulus and may include complement fixation and/or ADCC (antibody-dependent cellular cytotoxicity) induction and/or ADCP (antibody-dependent cellular phagocytosis).

In some embodiments, a biological activity of an Fc polypeptide is the ability to bind FcRn. In some embodiments, a biological activity of an Fc polypeptide is the ability to bind C1q. In some embodiments, a biological activity of an Fc polypeptide is the ability to bind CD16. In some embodiments, a biological activity of an Fc polypeptide is the ability to bind protein A.

The term “IgX Fe” means the Fc region is derived from a particular antibody isotype (e.g., IgG, IgA, IgD, IgE, IgM, etc.), where “X” denotes the antibody isotype. Thus, “IgG Fc” denotes the Fc region of a γ chain, “IgA Fc” denotes the Fc region of an a chain, “IgD Fc” denotes the Fc region of a δ chain, “IgE Fc” denotes the Fc region of an F chain, “IgM Fc” denotes the Fc region of a μ chain, etc. In some embodiments, the IgG Fc region comprises CH1, hinge, CH2, CH3, and CL1. “IgX-N-Fc” denotes that the Fc region is derived from a particular subclass of antibody isotype (such as canine IgG subclass A, B, C, or D; or feline IgG subclass 1, 2a, or 2b), where “N” denotes the subclass. In some embodiments, IgX Fc or IgX-N-Fc regions are derived from a companion animal, such as a dog or a cat. In some embodiments, IgG Fc regions are isolated from canine γ heavy chains, such as IgG-A, IgG-B, IgG-C, or IgG-D. In some instances, IgG Fc regions are isolated from feline γ heavy chains, such as IgG1, IgG2a, or IgG2b. Antibodies comprising an Fc region of IgG-A, IgG-B, IgG-C, or IgG-D may provide for higher expression levels in recombination production systems.

The terms “IgX Fe” and “IgX Fc polypeptide” include wild-type IgX Fc polypeptides and variant IgX Fc polypeptides, unless indicated otherwise.

In some embodiments, a variant IgG Fc polypeptide comprises a variant IgG Fc polypeptide of a companion animal species. In some embodiments, a variant IgG Fc polypeptide comprises a variant canine IgG Fc polypeptide or a feline IgG Fc polypeptide. In some embodiments, a variant IgG Fc polypeptide (e.g., a variant canine IgG-A Fc polypeptide, a variant canine IgG-C Fc polypeptide, or a variant canine IgG-D Fc polypeptide, variant feline IgGla Fc polypeptide, variant feline IgG1b Fc polypeptide, or variant feline IgG2 Fc polypeptide) has an activity that the reference (e.g., wild-type) polypeptide substantially lacks.

An antibody may be modified to extend or shorten its half-life. In some embodiments involving a higher dose of antibody, a shorter half-life may be desirable for acute treatment. In some embodiments involving a lower dose of antibody, a longer half-life may be desirable for prolonged treatment. For example, as discussed below, mutations in IgG Fe that affect FcRn interactions may be introduced.

In some embodiments, a parvovirus antibody comprises a wild-type or variant IgG Fc having complement fixation activity (or complement-dependent cytotoxicity (CDC)). In some embodiments, a parvovirus antibody comprises a wild-type or variant IgG Fc having antibody-dependent cellular cytotoxicity (ADCC) activity. In some embodiments, a parvovirus antibody comprises a wild-type or variant IgG Fc having antibody-dependent cellular phagocytosis (ADCP) activity. In some embodiments, a parvovirus antibody comprises a wild-type or variant IgG Fc having complement fixation activity and/or ADCC activity and/or ADCP activity. IgG Fc polypeptides may be modified to have an effector-function or to have an enhanced effector function.

In some embodiments, a parvovirus antibody comprises a wild-type or variant IgG Fe the binds to canine FcRn at low pH. In some embodiments, a parvovirus comprises a wild-type or variant IgG Fe that binds to C1q. In some embodiments, a parvovirus comprises a wild-type or variant IgG Fe that binds to CD16. In some embodiments, a parvovirus comprises a variant IgG Fc comprising one or more afucosylated glycan.

In some embodiments, a variant IgG Fe (e.g., a variant canine IgG Fc polypeptide or a variant feline IgG Fc polypeptide) has modified FcRn binding affinity compared to a reference polypeptide. In some embodiments, a variant IgG Fc has increased FcRn binding affinity at an acidic pH (e.g., at a pH in the range of from about 5.0 to about 6.5, such as at a pH of about 5.0, a pH of about 5.5, a pH of about 6.0, or a pH of about 6.5) compared to a reference polypeptide. Exemplary variant IgG Fc polypeptides having increased FcRn binding affinity are disclosed in WO 2020/082048, which is incorporated by reference herein in its entirety.

The term “affinity” means the strength of the sum-total of noncovalent interactions between a single binding site of a molecule (for example, an antibody) and its binding partner (for example, an antigen). The affinity of a molecule X for its partner Y can generally be represented by the dissociation constant (KD). Affinity can be measured by common methods known in the art, such as, for example, immunoblot, ELISA KD, KinEx A, biolayer interferometry (BLI), or surface plasmon resonance devices.

The terms “KD,” “Kd,” “Kd” or “Kd value” as used interchangeably to refer to the equilibrium dissociation constant of an antibody-antigen interaction. In some embodiments, the Kd of the antibody is measured by using biolayer interferometry assays using a biosensor, such as an Octet® System (Pall ForteBio LLC, Fremont, Calif.) according to the supplier's instructions. Briefly, biotinylated antigen is bound to the sensor tip and the association of antibody is monitored for ninety seconds and the dissociation is monitored for 600 seconds. The buffer for dilutions and binding steps is 20 mM phosphate, 150 mM NaCl, pH 7.2. A buffer only blank curve is subtracted to correct for any drift. The data are fit to a 2:1 binding model using ForteBio data analysis software to determine association rate constant (kon), dissociation rate constant (koff), and the Kd. The equilibrium dissociation constant (Kd) is calculated as the ratio of koff/kon. The term “kon” refers to the rate constant for association of an antibody to an antigen and the term “koff” refers to the rate constant for dissociation of an antibody from the antibody/antigen complex.

The term “binds” to an antigen or epitope is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA KD, KinEx A, biolayer interferometry (BLI), surface plasmon resonance devices, or etc.

“Wild-type” refers to a non-mutated version of a polypeptide that occurs in nature, or a fragment thereof. A wild-type polypeptide may be produced recombinantly.

A “variant” means a biologically active polypeptide having at least about 50% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide.

In some embodiments, a variant has at least 1, 2, 3, 4, or 5 amino acids substituted by a different amino acid.

In some embodiments, a variant has at least about 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide. In some embodiments, a variant has at least about 50% sequence identity, at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 97% sequence identity, at least about 98% sequence identity, or at least about 99% sequence identity with the sequence of the reference nucleic acid or polypeptide.

As used herein, “position corresponding to position n,” wherein n is any number, refers to an amino acid position of a subject polypeptide that aligns with position n of a reference polypeptide after aligning the amino acid sequences of the subject and reference polypeptides and introducing gaps. Alignment for purposes of whether a position of a subject polypeptide corresponds with position n of a reference polypeptide can be achieved in various ways that are within the skill in the art, for instance, using publicly available computer software.

A “point mutation” is a mutation that involves a single amino acid residue. The mutation may be the loss of an amino acid, substitution of one amino acid residue for another, or the insertion of an additional amino acid residue.

An “amino acid substitution” refers to the replacement of one amino acid in a polypeptide with another amino acid. In some embodiments, an amino acid substitution is a conservative substitution. Nonlimiting exemplary conservative amino acid substitutions are shown in Table 2. Amino acid substitutions may be introduced into a molecule of interest and the products screened for a desired activity, for example, retained/improved antigen binding, decreased immunogenicity, or improved ADCC or CDC or enhanced pharmacokinetics.

Non-conservative substitutions will entail exchanging a member of one of these classes with another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters or enhancers) that regulate the expression of the polypeptide of interest, or one or more selectable marker genes (such as, for example, antibiotic resistance genes and genes that can be used in colorimetric assays, for example, 0-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.” In some embodiments, the parvovirus antibody is purified using chromatography, such as size exclusion chromatography, ion exchange chromatography, protein A column chromatography, hydrophobic interaction chromatography, and CHT chromatography.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

The term “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 15% 20%, 25%, 30%, 35%, 40%, 45%, 50%, 60%, 70%, 80%, 90%, or 100% compared to the reference value.

In some embodiments, an parvovirus antibody may reduce parvovirus titers in a canine or a feline by at least 10%, at least 15%, at least 20%, at least 25%, at least 30%, at least 35%, at least 40%, at least 45%, at least 50%, at least 60%, at least 70%, at least 80%, at least 90%, or 100% compared to parvovirus titers in the absence of the antibody, as measured by a Hemagglutination Inhibition (HI) assay or Virus Neutralization (VN) assay. In some embodiments, the reduction in parvovirus titer is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog, cat, or horse. In other embodiments, particularly where the antibody is highly stable to thermal and oxidative denaturation, the pharmaceutical composition can be stored as a liquid, i.e., as an aqueous composition, which may be administered directly, or with appropriate dilution, to the dog, cat, or horse. A lyophilized composition can be reconstituted with sterile Water for Injection (WFI). Anti-bacterial agents (e.g., bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

In some embodiments, the pharmaceutically acceptable carrier or the pharmaceutical composition has a pH of from 5.0 to 6.2, from 5.0 to 6.0, or from 5.3 to 5.7. In some embodiments, the pharmaceutical carrier is phosphate buffered saline, pH 7.2. In some embodiments, the pharmaceutical carrier is 50 mM NaCitrate pH 7, 150 mM NaCl.

In some embodiments, the pharmaceutically acceptable carrier or a pharmaceutical composition comprises an anti-bacterial agent.

The antibodies or pharmaceutical compositions comprising the antibodies of the invention may be useful for providing passive immunity against infection with parvovirus and/or treating a parvoviral infection. As used herein, a “parvoviral infection” means a condition associated with, caused by, or characterized by, a parvoviral infection. Such conditions include, but are not limited to, infections confirmed by cage-side ELISA tests, hemagglutination assay (HA), histopathology, virus isolation or virus titers, or PCR. Infections with parvovirus often include fever, vomiting, diarrhea, lymphopenia, dehydration, and/or secondary septicemia.

As used herein, “treatment” is an approach for obtaining beneficial or desired clinical results. “Treatment” as used herein, covers any administration or application of a therapeutic for disease in a subject, such as a mammal, including a human and a companion animal (e.g., a canine or feline). For purposes of this disclosure, beneficial or desired clinical results include, but are not limited to, any one or more of: alleviation of one or more symptoms, prevention of mortality, diminishment of extent and severity of disease, preventing or delaying spread of disease, eliminating or shorting duration of viral shedding, preventing or delaying recurrence of disease, preventing or decreasing viral cytopathic effects, delay or slowing of disease progression, amelioration of the disease state, inhibiting the disease or progression of the disease, resolution of clinical signs of disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In some embodiments, a parvovirus antibody or pharmaceutical compositions comprising it can be utilized in accordance with the methods herein to provide passive immunity against infection parvovirus and/or treat parvoviral infections. In some embodiments, a parvovirus antibody or pharmaceutical compositions is administered to subject, such as a companion animal (e.g., a canine or a feline) or a human to provide passive immunity against infection with parvovirus and/or treat an a parvoviral infection.

A “therapeutically effective amount” of a substance/molecule, agonist or antagonist may vary according to factors such as the type of disease to be treated, the disease state, the immune status of the individual subject, the virulent viral load encountered, the severity and extent of viremia, the severity and course of the disease, the type of therapeutic purpose, any previous therapy, the clinical history, the response to prior treatment, the maternally-derived antibody passive transfer status, the previous immunization status of the individual animal, the discretion of the attending veterinarian, age, sex, and weight of the subject, and the ability of the substance/molecule, agonist or antagonist to elicit a desired response in the subject. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, a parvovirus antibody or pharmaceutical composition comprising a parvovirus antibody is administered parenterally, by subcutaneous administration, intravenous infusion, or intramuscular injection. In some embodiments, a parvovirus antibody or pharmaceutical composition comprising a parvovirus antibody is administered as a single dose or multiple dose bolus injection. In some embodiments, a parvovirus antibody or pharmaceutical composition comprising a parvovirus antibody is administered by an intramuscular, an intravenous, an intraperitoneal, an intracerebrospinal, a subcutaneous, an intra-arterial, an intrasynovial, an intrathecal, or an inhalation route.

Parvovirus antibodies described herein may be administered in an amount in the range of 0.01 mg/kg body weight to 100 mg/kg body weight per dose. In some embodiments, parvovirus antibodies may be administered in an amount in the range of 0.5 mg/kg body weight to 50 mg/kg body weight per dose. In some embodiments, parvovirus antibodies may be administered in an amount in the range of 0.1 mg/kg body weight to 10 mg/kg body weight per dose. In some embodiments, parvovirus antibodies may be administered in an amount in the range of 0.1 mg/kg body weight to 100 mg/kg body weight per dose. In some embodiments, parvovirus antibodies may be administered in an amount in the range of 1 mg/kg body weight to 10 mg/kg body weight per dose. In some embodiments, parvovirus antibodies may be administered in an amount in the range of 0.5 mg/kg body weight to 100 mg/kg body, in the range of 1 mg/kg body weight to 100 mg/kg body weight, in the range of 5 mg/kg body weight to 100 mg/kg body weight, in the range of 10 mg/kg body weight to 100 mg/kg body weight, in the range of 20 mg/kg body weight to 100 mg/kg body weight, in the range of 50 mg/kg body weight to 100 mg/kg body weight, in the range of 1 mg/kg body weight to 10 mg/kg body weight, in the range of 5 mg/kg body weight to 10 mg/kg body weight, in the range of 0.5 mg/kg body weight to 10 mg/kg body weight, in the range of 0.01 mg/kg body weight to 0.5 mg/kg body weight, in the range of 0.01 mg/kg body weight to 0.1 mg/kg body weight, or in the range of 5 mg/kg body weight to 50 mg/kg body weight. In some embodiments, parvovirus antibodies may be administered in an amount of 0.5 mg/kg body weight.

A parvovirus antibody or a pharmaceutical composition comprising a parvovirus antibody can be administered to a subject, such as a human or companion animal (e.g., a canine or a feline) as a single dose, at one time or over a series of treatments. For example, a parvovirus antibody or a pharmaceutical composition comprising a parvovirus antibody may be administered at least once, more than once, at least twice, at least three times, at least four times, or at least five times.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

In some embodiments, the dose is administered to a subject, such as a human or companion animal (e.g., a canine or a feline) less than 1 week of age, less than 2 weeks of age, less than 3 weeks of age, less than 4 weeks of age, less than 5 weeks of age, less than 6 weeks of age, less than 6 weeks of age, less than 7 weeks of age, less than 8 weeks of age, less than 9 weeks of age, less than 10 weeks of age, less than 11 weeks of age, less than 12 weeks of age, less than 6 months of age, between 0 and 12 weeks of age, between 0 and 10 weeks of age, between 0 and 8 weeks of age, between 0 and 6 weeks of age, between 0 and 4 weeks of age, between 0 and 2 weeks of age, between 4 and 12 weeks of age, between 6 and 12 weeks of age, between 10 and 12 weeks of age, between 4 weeks and 6 months of age, between 2 months and 6 months of age, between 4 months and 6 months of age, between 6 months and 1 year of age, greater than 13 weeks of age, or greater than 1 year of age.

It may be advantageous to deliver the parvovirus antibody or a nucleic acid encoding the parvovirus antibody to an infant subject prenatally and/or postnatally to provide passive immunity against parvovirus infection. In some embodiments, the parvovirus antibody is administered to a pregnant or nursing maternal subject, such as a human or companion animal (e.g., a canine or a feline). In some embodiments, the parvovirus antibody is administered to the placenta of a pregnant subject.

In some embodiments, a method of providing passive immunity in an infant subject against infection with a canine or feline parvovirus comprises administering to a pregnant or nursing maternal subject a therapeutically effective amount of a monoclonal antibody that binds to the canine or feline parvovirus. In some embodiments the parvovirus antibody is administered to the placenta of a pregnant subject. In some embodiments, the parvovirus antibody is administered to a nursing subject.

Provided herein are methods of using the parvovirus antibodies, polypeptides and polynucleotides for detection, diagnosis and monitoring of a parvoviral infection. Provided herein are methods of determining whether a subject will respond to parvovirus antibody therapy. In some embodiments, the method comprises virus serum neutralization. In some embodiments, the method comprises detecting whether the subject has cells that express parvovirus using a parvovirus antibody. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the antibodies or polypeptides described herein are an appropriate treatment for the subject.

In some embodiments, the sample is a biological sample. The term “biological sample” means a quantity of a substance from a living thing or formerly living thing. In some embodiments, the biological sample is a swab containing cellular debris, cell or cell/tissue lysate. In some embodiments, the biological sample includes, but is not limited to, blood, (for example, whole blood), plasma, serum, urine, synovial fluid, lymphatic tissue and epithelial cells.

In some embodiments, the cells or cell/tissue lysate are contacted with a parvovirus antibody and the binding between the antibody and the cell is determined. When the test cells show binding activity as compared to a reference cell of the same tissue type, it may indicate that the subject would benefit from treatment with a parvovirus antibody. In some embodiments, the test cells are from tissue of a subject, such as a human or companion animal (e.g., a canine or a feline).

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (for example 125I, 131I, 35S, 3H, or 32P), enzymes (for example, alkaline phosphatase, horseradish peroxidase, luciferase, or b-galactosidase), fluorescent moieties or proteins (for example, fluorescein, rhodamine, phycoerythrin, GFP, or BFP), or luminescent moieties (for example, QDOT™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art. In some embodiments, the parvovirus antibodies need not be labeled, and the presence thereof can be detected using a second labeled antibody which binds to the first parvovirus antibody. In some embodiments, the parvovirus antibody can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). The parvovirus antibodies and polypeptides can also be used for in vivo diagnostic assays, such as in vivo imaging. Generally, the antibody or the polypeptide is labeled with a radionuclide (such as 111In, 99Tc, 14C, 131I, 125I, 13H, or any other radionuclide label, including those outlined herein) so that the cells or tissue of interest can be localized using immunoscintiography. The antibody may also be used as staining reagent in pathology using techniques well known in the art.

In some embodiments, a first antibody is used for a diagnostic and a second antibody is used as a therapeutic. In some embodiments, the first and second antibodies are different. In some embodiments, the first and second antibodies can both bind to the antigen at the same time, by binding to separate epitopes.

The following examples illustrate some aspects of the disclosure and are not intended in any way to limit the disclosure.

### BRIEF DESCRIPTION OF THE DRAWINGS

FIG. 1A and FIG. 1B show that three beagles were highly immunized with canine parvovirus vaccine (CPV2a), spleens were collected, and canine antibody libraries were prepared from spleen B cells, and the antibody clones obtained as a result of bio-panning three times using ELISA coated with the VP2 protein of canine parvovirus (CPV 2c), these antibody sequences were positively confirmed as a result of FA reaction in A72 cells infected with canine parvovirus.

FIG. 2A-FIG. 2C are results of confirming the ability to inhibit canine parvovirus proliferation by concentration of antibody therapeutic candidate strains.

### DETAILED DESCRIPTION OF EXEMPLARY EMBODIMENTS

Objects and advantages of the present invention, and technical configurations for achieving them, will become apparent with reference to the embodiments described in detail below in conjunction with the accompanying drawings. In the description of the present invention, when it is determined that a specific description of a known function or configuration may unnecessarily obscure the gist of the present invention, the detailed description thereof will be omitted. And the following terms are defined terms in consideration of the donation in the present invention, which may vary according to the user, the intent or practice of the operator, etc.

However, the present invention is not limited to the embodiments disclosed below, but may be embodied in various different forms. The examples are merely provided to complete the disclosure of the present invention and to fully illuminate the scope of the invention to those skilled in the art, and the invention is only defined by the scope of the claims. The definition should therefore be made on the basis of the disclosure throughout this specification.

Hereinafter, examples of the present invention will be described in detail.

An antibody library was constructed to prepare parvovirus antibodies according to an embodiment of the present invention, and specifically, a canine parvovirus antibody library obtained from immune memory B cells in dogs immunized with canine parvovirus antigens using a phage display method was constructed (phage display biopanning).

To construct a parvovirus antibody library, 3 beagles were highly immunized with canine parvovirus vaccine (CPV2a), and spleens were collected and canine antibody molecular PCR was performed on splenic B cells (Ref. Mason N J et al., 2011).

After constructing an scFv by connecting the amplified heavy chain (HC) and light chain (LC) with a linker, and producing an antibody library by method of the phage display. Antibody clones produced by the phage display method were biopanned three times using ELISA coated with the VP2 protein of recombinant protein canine parvovirus (CPV 2c) to select those with good reactivity.

### Example 1: Selection and Evaluation of Recombinant Antibodies Against Canine Parvovirus

### 1) Construction of Canine Parvovirus Antibody Library and Selection of Antibody Candidates

### (A) Production of Antibody Library by Phage Display Method

Antibodies produced by the phage display method were biopanned three times, and then a total of 223 antibody clones were selected by ELISA coated with canine parvovirus (CPV 2c) VP2 recombinant protein (expressed in insect cells).

(B) In Order to Confirm the Reactivity of 223 Selected Clones with Canine Parvovirus, A72 Cells Infected with Canine Parvovirus (CPV 2c) were Subjected to FA Reaction, and 43 Clones were Positive.

Among 43 clones, 11 scFv FRs (Framework regions) and CDRs (Complementarity-determining regions) gene sequences were obtained.

TABLE 1

| | Phage | Confirmation of | | |
|---|---|---|---|---|
| | display | reactivity with | Positive scFv | Selected |
| | Biopanning | intracellular CPV | sequence(s) | scFv(s) |
| amount | 223 | 43 | 11 | 2 |

As a result of comparing the amino acid sequences of the 11 antibody clones obtained, the VH and VL domains consisted of 4 framework regions (FRs) and 3 complementarity-determining regions (CDRs), respectively. In particular, it was confirmed that the CDR3 portion of the VH domain had the greatest diversity. In the reactivity test with canine parvovirus using periplasmic sup, the final two clones with potential and good expression were selected.

### (C) Production of Additional Clones Through Affinity Maturation Using the Final Two Selected Clones

PCR was carried out as it is in the heavy chain variable region of the selected antibody. Then, phage display was performed by linking with canine light chain variable region pool DNA. Specifically, the basic VH and various VLs were attached to create an antibody pool with only VLs different.

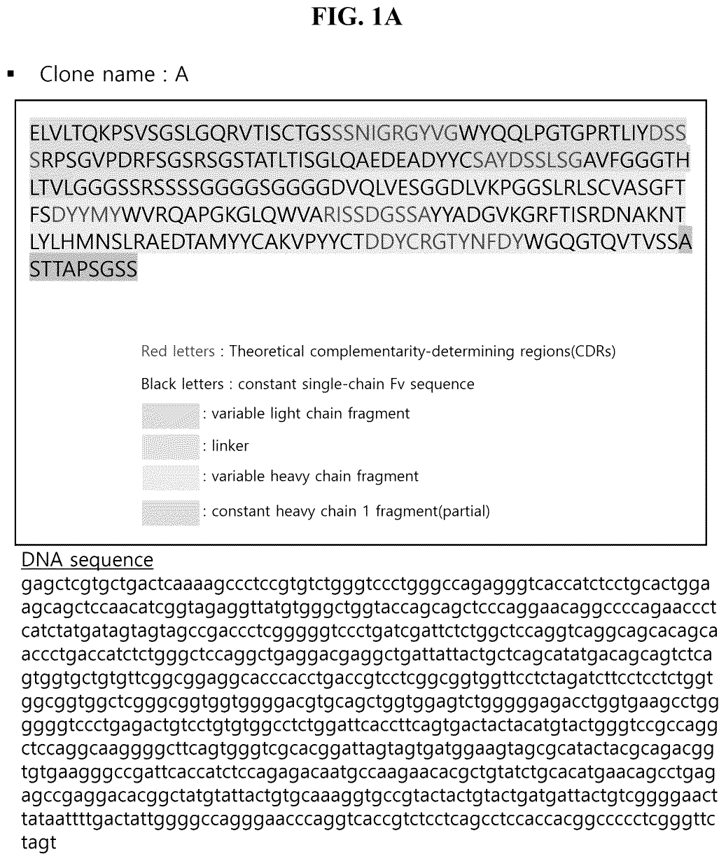

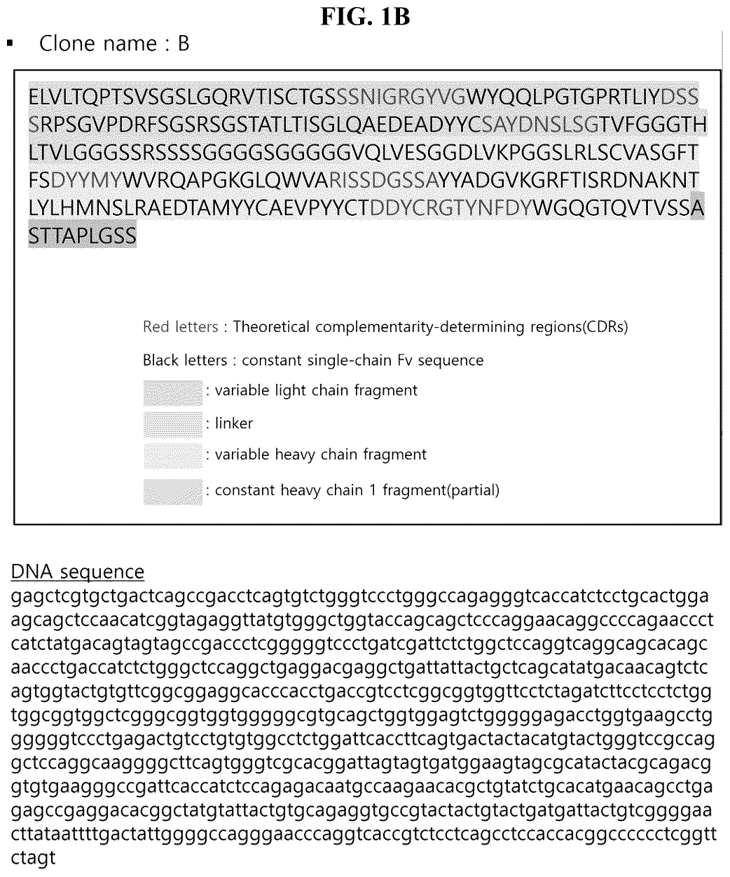

Next, clones highly reactive with canine parvovirus were selected through the ELISA method. Among the 5 clones with good reactivity, the final 2 clones were selected after sequencing. FIG. 1A and FIG. 1B show that 3 beagle dogs were highly immunized with canine parvovirus vaccine (CPV2a), and spleens were collected to construct canine antibody libraries from spleen B cells. FIG. 1A and FIG. 1B show the positively confirmed antibody sequences. The sequences were obtained by FA reaction in A72 cells infected with canine parvovirus among the antibody clones obtained as a result of bio-panning three times using ELISA coated with the VP2 protein of canine parvovirus (CPV 2c). Table 2 shows the antibody sequences of the selected clones.

TABLE 2

| SEQ | |
|---|---|
| ID | |
| NO: | antibody sequence |
| 1 | ELVLTQKPSVSGSLGQRVTISCTGSSSNIGRGYVGWYQQLPGTGPR |
| | TLIYDSSSRPSGVPDRFSGSRSGSTATLTISGLQAEDEADYYCSAY |
| | DSSLSGAVFGGGTHLTVLGGGSSRSSSSGGGGSGGGGDVQLVESGG |
| | DLVKPGGSLRLSCVASGFTFSDYYMYWVRQAPGKGLQWVARISSDG |
| | SSAYYADGVKGRFTISRDNAKNTLYLHMNSLRAEDTAMYYCAKVPY |
| | YCTDDYCRGTYNFDYWGQGTQVTVSSASTTAPSGSS |
| 2 | ELVLTQPTSVSGSLGQRVTISCTGSSSNIGRGYVGWYQQLPGTGPR |
| | TLIYDSSSRPSGVPDRFSGSRSGSTATLTISGLQAEDEADYYCSAY |
| | DNSLSGTVFGGGTHLTVLGGGSSRSSSSGGGGSGGGGGVQLVESGG |
| | DLVKPGGSLRLSCVASGFTFSDYYMYWVRQAPGKGLQWVARISSDG |
| | SSAYYADGVKGRFTISRDNAKNTLYLHMNSLRAEDTAMYYCAEVPY |
| | YCTDDYCRGTYNFDYWGQGTQVTVSSASTTAPLGSS |

TABLE 3

| Antibody | domain | sequence | SEQ. No. |
|---|---|---|---|
| A | Heavy chain variable | DVQLVESGGDLVKPGGSLRLSCVASGFT | 12 |
| | region | FSDYYMYWVRQAPGKGLQWVARISSD | |
| | | GSSAYYADGVKGRFTISRDNAKNTLYL | |
| | | HMNSLRAEDTAMYYCAKVPYYCTDDY | |
| | | CRGTYNFDYWGQGTQVTVSS | |
| | HC-FR1 | DVQLVESGGDLVKPGGSLRLSCVASGFT | 13 |
| | | FS | |
| | CDR-H1 | DYYMY | 14 |
| | HC-FR2 | WVRQAPGKGLQWVA | 15 |
| | CDR-H2 | RISSDGSSA | 16 |
| | HC-FR3 | YYADGVKGRFTISRDNAKNTLYLHMNS | 17 |
| | | LRAEDTAMYYCAKVPYYCT | |
| | CDR-H3 | DDYCRGTYNFDY | 18 |
| | HC-FR4 | WGQGTQVTVSS | 19 |
| | Light chain variable | ELVLTQKPSVSGSLGQRVTISCTGSSSNI | 20 |
| | region | GRGYVGWYQQLPGTGPRTLIYDSSSRPS | |
| | | GVPDRFSGSRSGSTATLTISGLQAEDEAD | |
| | | YYCSAYDSSLSGAVFGGGTHLTVL | |
| | LC-FR1 | ELVLTQKPSVSGSLGQRVTISCTGS | 21 |
| | CDR-L1 | SSNIGRGYVG | 22 |
| | LC-FR2 | WYQQLPGTGPRTLIY | 23 |
| | CDR-L2 | DSSS | 24 |
| | LC-FR3 | RPSGVPDRFSGSRSGSTATLTISGLQAED | 25 |
| | | EADYYC | |
| | CDR-L3 | SAYDSSLSG | 26 |
| | LC-FR4 | AVFGGGTHLTVL | 27 |
| B | Heavy chain variable | VQLVESGGDLVKPGGSLRLSCVASGFTF | 28 |
| | region | SDYYMYWVRQAPGKGLQWVARISSDG | |
| | | SSAYYADGVKGRFTISRDNAKNTLYLH | |
| | | MNSLRAEDTAMYYCAEVPYYCTDDYC | |
| | | RGTYNFDYWGQGTQVTVSS | |
| | HC-FR1 | VQLVESGGDLVKPGGSLRLSCVASGFTF | 29 |
| | | S | |
| | CDR-H1 | DYYMY | 30 |
| | HC-FR2 | WVRQAPGKGLQWVA | 31 |
| | CDR-H2 | RISSDGSSA | 32 |
| | HC-FR3 | YYADGVKGRFTISRDNAKNTLYLHMNS | 33 |
| | | LRAEDTAMYYCAEVPYYCT | |
| | CDR-H3 | DDYCRGTYNFDY | 34 |
| | HC-FR4 | WGQGTQVTVSS | 35 |
| | Light chain variable | ELVLTQPTSVSGSLGQRVTISCTGSSSNIG | 36 |
| | region | RGYVGWYQQLPGTGPRTLIYDSSSRPSG | |
| | | VPDRFSGSRSGSTATLTISGLQAEDEADY | |
| | | YCSAYDNSLSGTVFGGGTHLTVL | |
| | LC-FR1 | ELVLTQPTSVSGSLGQRVTISCTGS | 37 |
| | CDR-L1 | SSNIGRGYVG | 38 |
| | LC-FR2 | WYQQLPGTGPRTLIY | 39 |
| | CDR-L2 | DSSS | 40 |
| | LC-FR3 | RPSGVPDRFSGSRSGSTATLTISGLQAED | 41 |
| | | EADYYC | |
| | CDR-L3 | SAYDNSLSG | 42 |
| | LC-FR4 | TVFGGGTHLTVL | 43 |

### Example 2: Selection and Characterization of Canine Parvovirus Antibody Treatment Candidates

Two strains were selected (A and B) as final candidates for canine parvovirus antibody treatment, and the two clones were cloned into a mammalian expression vector expressing Human Fc and expressed in 293T cells.

Table 4 below shows the comparison results of antibody therapeutics HI and neutralizing antibody titer.

TABLE 4

| Minibody | HI-CPV2(Vaccine strain) | VN-CPV2a(Outdoor strain) |
|---|---|---|
| A | 2¹⁶(65,536) | 2¹⁵(32,768) |
| B | 2¹³(8,192) | 2¹⁵(32,768) |

### Example 3: Analysis of Efficacy and Effectiveness of Candidate Antibody Therapeutics

Next, a comparative analysis of hemagglutination inhibition test (HI) and neutralizing antibody titer (VN) was performed.
(A) In the hemagglutination inhibition test on canine parvovirus vaccine strains and outdoor strains (2a, 2b, 2c) of antibody treatment candidate strains (A, B), both clones showed antibody titers of 2¹³ or higher only in the vaccine strains. However, the neutralizing antibody titer showed an antibody titer of 2¹³ or higher in both the vaccine strain and the outdoor strain.

Highly immune serum (commercially available dog parvo treatment) used as a positive control showed an antibody titer of 2⁶ only in the vaccine strain in the blood aggregation inhibition test, and the vaccine strain and outdoor strain showed an antibody titer of 2⁶ or higher in the neutralization test.

Table 5 below shows the HI and neutralizing antibody titers of antibody therapeutic candidate strains.

TABLE 5

| | Hemagglutination inhibition test | | | | Neutralization test | | | |
|---|---|---|---|---|---|---|---|---|
| | CPV2 | | | | CPV2 | | | |
| | (Vaccine | | | | (Vaccine | | | |
| Minibody | strain) | CPV2a | CPV2b | CPV2c | strain) | CPV2a | CPV2b | CPV2c |
| A | 2¹⁶ | <2 | <2 | <2 | 2¹³ | 2¹⁵ | 2¹⁶ | 2¹⁶ |
| | (65, 536) | | | | (8, 192) | (32, 768) | (65, 536) | (65, 536) |
| B | 2¹³ | <2 | <2 | <2 | 2¹³ | 2¹⁵ | 2¹⁶ | 2¹⁶ |
| | (8, 192) | | | | (8, 192) | (32, 768) | (65, 536) | (65, 536) |
| Hyper | 2⁶ | <2 | <2 | <2 | 2⁶ | 2⁸ | 2⁸ | 2⁹ |
| serum | (64) | | | | (64) | (256) | (256) | (512) |

(B) As a result of examining the neutralizing antibody activity of antibody treatment candidate strains against feline parvovirus similar to canine parvovirus, it was confirmed that they had antibody activity of 2⁵ or higher against feline parvovirus (FPV).

Table 6 below shows the comparison results of canine parvovirus and feline parvovirus neutralizing antibody titers of antibody therapeutic candidate strains.

TABLE 6

Neutralization test

| Minibody | FPV | CPV2a |
|---|---|---|
| A | 2⁷(128) | 2¹⁵(32,768) |
| B | 2⁵(32) | 2¹⁵(32,768) |
| HYPER SERUM | 2⁷(128) | 2⁸(256) |

### Example 4: Investigation of Canine Parvovirus Growth Inhibitory Ability of Antibody Therapeutic Candidates

In order to confirm the ability of antibody A and antibody B to inhibit canine parvovirus proliferation, antibody treatment candidate strains (A-Fc, B-Fc), canine parvovirus hyperimmune serum, and canine parvovirus genotype were mixed 1:1 and CRFK cells were mixed. After 1 hour, the sensitized mixture was removed and incubated for 72 hours.

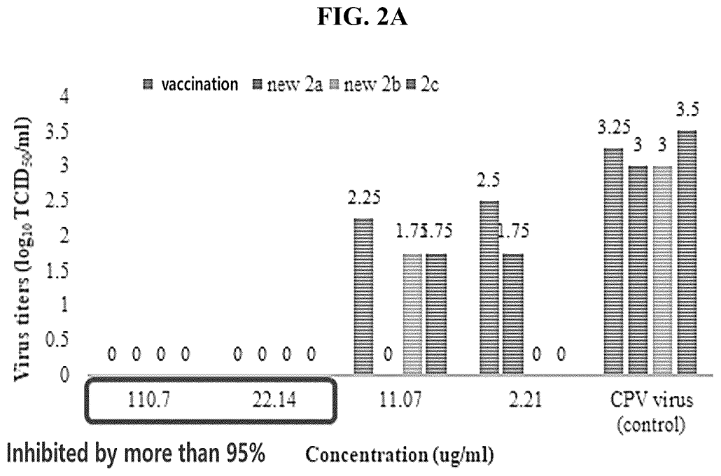

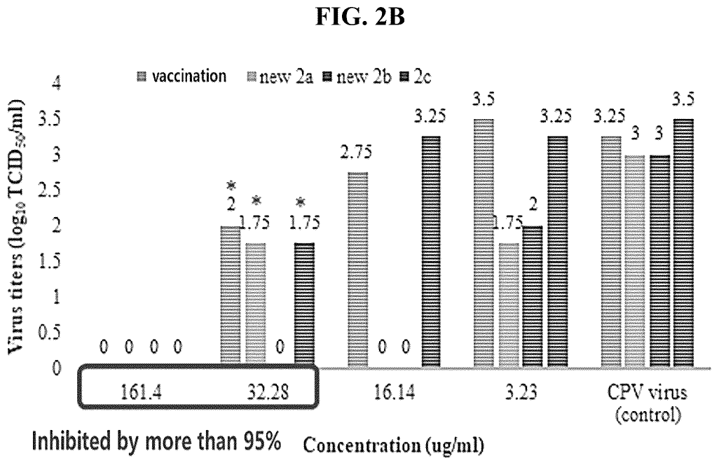

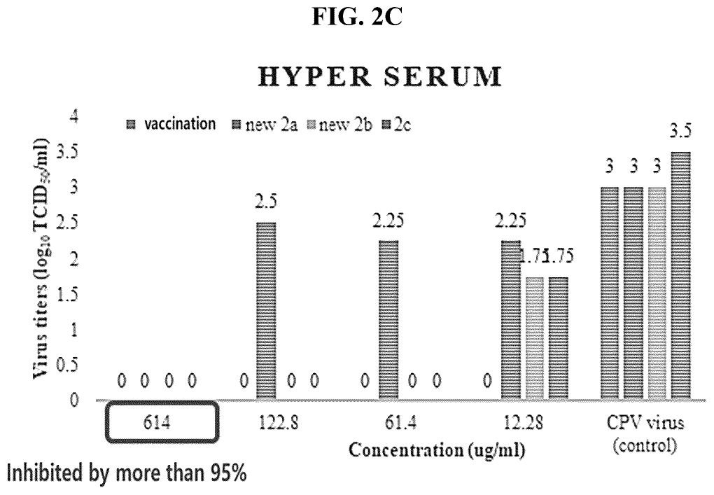

After culturing, the supernatant was titrated in CRFK cells, and the cells were fixed and confirmed for virus infection through fluorescence staining. FIG. 2A-FIG. 2C are results of confirming the ability to inhibit canine parvovirus proliferation by concentration of antibody therapeutic candidate strains. As can be seen in FIGS. 2A-2C, in the case of antibody therapeutic candidate strains (A-Fc, B-Fc) and hyperimmune serum, it was confirmed that the concentration inhibited proliferation by 95% or more.

In order to confirm the virus proliferation inhibitory ability according to the sensitization time zone of candidate antibody therapeutics and canine parvovirus (CPV 2a, outdoor strain), it was added 2 hours before infection, simultaneously with infection, 3 hours after infection, and 5 hours after infection. They were incubated for 72 hours, respectively.

TABLE 7
Antibody

| therapeutic | concentration | prior to | during | post infection | |
|---|---|---|---|---|---|
| candidate | (mg/ml) | infection | infection | 3 hr | 5 hr |
| A | 1 | 10^{2.5} | 10^{2.5} | 10^{3.5} | 10^{4.5} |
| | 0.1 | 10^{3.5} | 10^{3.5} | 10^{3.5} | 10^{4.5} |
| B | 1 | 10^{2.0} | 10^{2.5} | 10^{3.5} | 10^{4.5} |
| | 0.1 | 10^{2.5} | 10^{3.5} | 10^{3.5} | 10^{4.5} |
| HYPER | 1 | — | — | 10^{3.5} | 10^{4.5} |
| SERUM | 0.1 | 10^{3.5} | 10^{3.5} | 10^{3.5} | 10^{4.5} |

a; negative
Potivite control: CPV 2a: 10^{4.5} TCIT₅₀/ml

As a result of confirming the ability to inhibit the growth of canine parvovirus at each time of addition of antibody treatment candidate strains, it was confirmed that antibody A, antibody B, and high immune serum inhibited virus proliferation when administered before and at the same time as inoculation.

When the titer was measured 3 hours and 5 hours after viral infection, both showed titers similar to those of the positive control, and it was confirmed that the inhibitory effect of the treatment administration was lowered after virus proliferation.

In order to confirm the safety of the parvovirus antibody treatment candidate strains (A, B) before application to the target animal, Balb/c mice were inoculated intraperitoneally (100 ug/head) by 5 heads each, and body temperature and weight changes were observed, but it was confirmed that there was no abnormality.

As a result of constructing a library of canine parvovirus antibody therapeutics as in the above example and examining the canine parvovirus proliferation inhibitory ability of candidate antibody therapeutics, it was confirmed that both candidates inhibited the growth of canine parvovirus compared to the positive control group. There was no difference in growth inhibitory ability according to canine parvovirus genotype, and although the proliferation inhibitory concentration differed from clone to clone, it was confirmed that it was effective up to 0.1 mg/ml.

As a result of comparing the inhibitory ability of anti-viral growth by the time of addition of antibody treatment candidate strains, it was confirmed that the anti-viral proliferation was inhibited when antibody A, antibody B, and high immune serum were administered before and at the same time as inoculation. However, when administered 3 hours or 5 hours after viral infection, the virus titer was not different from that of the positive control. Through this, it was confirmed that the inhibitory effect was lowered when the treatment was administered after viral proliferation.

The present invention can provide Parvovirus monoclonal antibodies to reduce or eliminate the severity of morbidity and mortality associated with CPV. The present invention can provide Parvovirus monoclonal antibodies to prevent CPV infection. The effects of the present invention are not limited to those mentioned above, and other effects not mentioned may be clearly understood by those skilled in the art.

In the present specification and drawings, preferred embodiments of the present invention have been disclosed, and although specific terms are used, these are only used in a general sense to easily explain the technical content of the present invention and help the understanding of the present invention. It is not intended to limit the scope. It will be apparent to those skilled in the art that other modifications based on the technical concept of the present invention can be performed in addition to the embodiments disclosed herein.

What is claimed is:

1. An isolated antibody that binds to parvovirus, wherein the antibody comprises:
a) a CDR-H1 comprising the amino acid sequence of SEQ ID NO: 14, a CDR-H2 comprising the amino acid sequence of SEQ ID NO: 16, a CDR-H3 comprising the amino acid sequence of SEQ ID NO: 18, and a CDR-L1 comprising the amino acid sequence of SEQ ID NO: 22, a CDR-L2 comprising the amino acid sequence of SEQ ID NO: 24, a CDR-L3 comprising the amino acid sequence of SEQ ID NO: 26, or
b) a CDR-H1 comprising the amino acid sequence of SEQ ID NO: 30, a CDR-H2 comprising the amino acid sequence of SEQ ID NO: 32, a CDR-H3 comprising the amino acid sequence of SEQ ID NO: 34, and a CDR-L1 comprising the amino acid sequence of SEQ ID NO: 38, a CDR-L2 comprising the amino acid sequence of SEQ ID NO: 40, a CDR-L3 comprising the amino acid sequence of SEQ ID NO: 42.

2. The isolated antibody of claim 1, wherein the antibody of a) comprises a HC-FR1 sequence of SEQ ID NO:13, a HC-FR2 sequence of SEQ ID NO:15, a HC-FR3 sequence of SEQ ID NO:17, a HC-FR4 sequence of SEQ ID NO:19, a LC-FR1 sequence of SEQ ID NO:21, a LC-FR2 sequence of SEQ ID NO:25, a LC-FR3 sequence of SEQ ID NO:25, a LC-FR4 sequence of SEQ ID NO:27.

3. The isolated antibody of claim 1, wherein the antibody of b) comprises a HC-FR1 sequence of SEQ ID NO:29, a HC-FR2 sequence of SEQ ID NO:31, a HC-FR3 sequence of SEQ ID NO:33, a HC-FR4 sequence of SEQ ID NO:35, a LC-FR1 sequence of SEQ ID NO:37, a LC-FR2 sequence of SEQ ID NO:39, a LC-FR3 sequence of SEQ ID NO:41, a LC-FR4 sequence of SEQ ID NO:43.

4. The antibody of claim 1, wherein the antibody is a chimeric antibody or a monoclonal antibody.

5. A pharmaceutical composition comprising the antibody of claim 1 and a pharmaceutically acceptable carrier.

6. An isolated nucleic acid encoding the antibody of claim 1.

7. A method of treating a canine parvoviral infection in a subject comprising administering to the subject a therapeutically effective amount of antibody that binds to the parvovirus.

8. The method of claim 7, wherein the subject is a canine or feline.

9. The method of claim 7, wherein the antibody is administered after the subject has exhibited at least one symptoms selected from fever, vomiting, diarrhea, lymphopenia, and septicemia.

10. A method of providing passive immunity in a subject against infection with a parvovirus comprising administering to the subject a therapeutically effective amount of the antibody of claim 1 that binds to the parvovirus.

11. The antibody of claim 2, wherein the antibody is a chimeric antibody or a monoclonal antibody.

12. The antibody of claim 3, wherein the antibody is a chimeric antibody or a monoclonal antibody.


SEQUENCE LISTING (43 sequences)

SEQ ID NO: 1 - protein, 266 aa, synthetic construct
ELVLTQKPSVSGSLGQRVTISCTGSSSNIGRGYVGWYQQLPGTGPRTLIYDSSSRPSGVPDRFSGSRSGSTATLTISGLQAEDEADYYCSAYDSSLSGAVFGGGTHLTVLGGGSSRSSSSGGGGSGGGGDVQLVESGGDLVKPGGSLRLSCVASGFTFSDYYMYWVRQAPGKGLQWVARISSDGSSAYYADGVKGRFTISRDNAKNTLYLHMNSLRAEDTAMYYCAKVPYYCTDDYCRGTYNFDYWGQGTQVTVSSASTTAPSGSS

SEQ ID NO: 2 - protein, 266 aa, synthetic construct
ELVLTQPTSVSGSLGQRVTISCTGSSSNIGRGYVGWYQQLPGTGPRTLIYDSSSRPSGVPDRFSGSRSGSTATLTISGLQAEDEADYYCSAYDNSLSGTVFGGGTHLTVLGGGSSRSSSSGGGGSGGGGGVQLVESGGDLVKPGGSLRLSCVASGFTFSDYYMYWVRQAPGKGLQWVARISSDGSSAYYADGVKGRFTISRDNAKNTLYLHMNSLRAEDTAMYYCAEVPYYCTDDYCRGTYNFDYWGQGTQVTVSSASTTAPLGSS

SEQ ID NO: 3 - DNA, 798 bp, synthetic construct
  source 1..798: mol_type = other DNA
gagctcgtgctgactcaaaagccctccgtgtctgggtccctgggccagagggtcaccatctcctgcactggaagcagctccaacatcggtagaggttatgtgggctggtaccagcagctcccaggaacaggccccagaaccctcatctatgatagtagtagccgaccctcgggggtccctgatcgattctctggctccaggtcaggcagcacagcaaccctgaccatctctgggctccaggctgaggacgaggctgattattactgctcagcatatgacagcagtctcagtggtgctgtgttcggcggaggcacccacctgaccgtcctcggcggtggttcctctagatcttcctcctctggtggcggtggctcgggcggtggtggggacgtgcagctggtggagtctgggggagacctggtgaagcctggggggtccctgagactgtcctgtgtggcctctggattcaccttcagtgactactacatgtactgggtccgccaggctccaggcaaggggcttcagtgggtcgcacggattagtagtgatggaagtagcgcatactacgcagacggtgtgaagggccgattcaccatctccagagacaatgccaagaacacgctgtatctgcacatgaacagcctgagagccgaggacacggctatgtattactgtgcaaaggtgccgtactactgtactgatgattactgtcggggaacttataattttgactattggggccagggaacccaggtcaccgtctcctcagcctccaccacggccccctcgggttctagt

SEQ ID NO: 4 - DNA, 798 bp, synthetic construct
  source 1..798: mol_type = other DNA
gagctcgtgctgactcagccgacctcagtgtctgggtccctgggccagagggtcaccatctcctgcactggaagcagctccaacatcggtagaggttatgtgggctggtaccagcagctcccaggaacaggccccagaaccctcatctatgacagtagtagccgaccctcgggggtccctgatcgattctctggctccaggtcaggcagcacagcaaccctgaccatctctgggctccaggctgaggacgaggctgattattactgctcagcatatgacaacagtctcagtggtactgtgttcggcggaggcacccacctgaccgtcctcggcggtggttcctctagatcttcctcctctggtggcggtggctcgggcggtggtgggggcgtgcagctggtggagtctgggggagacctggtgaagcctggggggtccctgagactgtcctgtgtggcctctggattcaccttcagtgactactacatgtactgggtccgccaggctccaggcaaggggcttcagtgggtcgcacggattagtagtgatggaagtagcgcatactacgcagacggtgtgaagggccgattcaccatctccagagacaatgccaagaacacgctgtatctgcacatgaacagcctgagagccgaggacacggctatgtattactgtgcagaggtgccgtactactgtactgatgattactgtcggggaacttataattttgactattggggccagggaacccaggtcaccgtctcctcagcctccaccacggcccccctcggttctagt

SEQ ID NO: 5 - protein, 19 aa, synthetic construct
GGGSSRSSSSGGGGSGGGG

SEQ ID NO: 6 - protein, 20 aa, synthetic construct
GGGSSRSSSSGGGGSGGGGG

SEQ ID NO: 7 - protein, 10 aa, synthetic construct
ASTTAPSGSS

SEQ ID NO: 8 - protein, 10 aa, synthetic construct
ASTTAPLGSS

SEQ ID NO: 9 - protein, 256 aa, synthetic construct
ELVLTQKPSVSGSLGQRVTISCTGSSSNIGRGYVGWYQQLPGTGPRTLIYDSSSRPSGVPDRFSGSRSGSTATLTISGLQAEDEADYYCSAYDSSLSGAVFGGGTHLTVLGGGSSRSSSSGGGGSGGGGDVQLVESGGDLVKPGGSLRLSCVASGFTFSDYYMYWVRQAPGKGLQWVARISSDGSSAYYADGVKGRFTISRDNAKNTLYLHMNSLRAEDTAMYYCAKVPYYCTDDYCRGTYNFDYWGQGTQVTVSS

SEQ ID NO: 10 - protein, 266 aa, synthetic construct; identical to SEQ ID NO: 1

SEQ ID NO: 11 - protein, 256 aa, synthetic construct
ELVLTQPTSVSGSLGQRVTISCTGSSSNIGRGYVGWYQQLPGTGPRTLIYDSSSRPSGVPDRFSGSRSGSTATLTISGLQAEDEADYYCSAYDNSLSGTVFGGGTHLTVLGGGSSRSSSSGGGGSGGGGGVQLVESGGDLVKPGGSLRLSCVASGFTFSDYYMYWVRQAPGKGLQWVARISSDGSSAYYADGVKGRFTISRDNAKNTLYLHMNSLRAEDTAMYYCAEVPYYCTDDYCRGTYNFDYWGQGTQVTVSS

SEQ ID NO: 12 - protein, 127 aa, synthetic construct
DVQLVESGGDLVKPGGSLRLSCVASGFTFSDYYMYWVRQAPGKGLQWVARISSDGSSAYYADGVKGRFTISRDNAKNTLYLHMNSLRAEDTAMYYCAKVPYYCTDDYCRGTYNFDYWGQGTQVTVSS

SEQ ID NO: 13 - protein, 30 aa, synthetic construct
DVQLVESGGDLVKPGGSLRLSCVASGFTFS

SEQ ID NO: 14 - protein, 5 aa, synthetic construct
DYYMY

SEQ ID NO: 15 - protein, 14 aa, synthetic construct
WVRQAPGKGLQWVA

SEQ ID NO: 16 - protein, 9 aa, synthetic construct
RISSDGSSA

SEQ ID NO: 17 - protein, 46 aa, synthetic construct
YYADGVKGRFTISRDNAKNTLYLHMNSLRAEDTAMYYCAKVPYYCT

SEQ ID NO: 18 - protein, 12 aa, synthetic construct
DDYCRGTYNFDY

SEQ ID NO: 19 - protein, 11 aa, synthetic construct
WGQGTQVTVSS

SEQ ID NO: 20 - protein, 110 aa, synthetic construct
ELVLTQKPSVSGSLGQRVTISCTGSSSNIGRGYVGWYQQLPGTGPRTLIYDSSSRPSGVPDRFSGSRSGSTATLTISGLQAEDEADYYCSAYDSSLSGAVFGGGTHLTVL

SEQ ID NO: 21 - protein, 25 aa, synthetic construct
ELVLTQKPSVSGSLGQRVTISCTGS

SEQ ID NO: 22 - protein, 10 aa, synthetic construct
SSNIGRGYVG

SEQ ID NO: 23 - protein, 15 aa, synthetic construct
WYQQLPGTGPRTLIY

SEQ ID NO: 24 - protein, 4 aa, synthetic construct
DSSS

SEQ ID NO: 25 - protein, 35 aa, synthetic construct
RPSGVPDRFSGSRSGSTATLTISGLQAEDEADYYC

SEQ ID NO: 26 - protein, 9 aa, synthetic construct
SAYDSSLSG

SEQ ID NO: 27 - protein, 12 aa, synthetic construct
AVFGGGTHLTVL

SEQ ID NO: 28 - protein, 126 aa, synthetic construct
VQLVESGGDLVKPGGSLRLSCVASGFTFSDYYMYWVRQAPGKGLQWVARISSDGSSAYYADGVKGRFTISRDNAKNTLYLHMNSLRAEDTAMYYCAEVPYYCTDDYCRGTYNFDYWGQGTQVTVSS

SEQ ID NO: 29 - protein, 29 aa, synthetic construct
VQLVESGGDLVKPGGSLRLSCVASGFTFS

SEQ ID NO: 30 - protein, 5 aa, synthetic construct
DYYMY

SEQ ID NO: 31 - protein, 14 aa, synthetic construct
WVRQAPGKGLQWVA

SEQ ID NO: 32 - protein, 9 aa, synthetic construct
RISSDGSSA

SEQ ID NO: 33 - protein, 46 aa, synthetic construct
YYADGVKGRFTISRDNAKNTLYLHMNSLRAEDTAMYYCAEVPYYCT

SEQ ID NO: 34 - protein, 12 aa, synthetic construct
DDYCRGTYNFDY

SEQ ID NO: 35 - protein, 11 aa, synthetic construct
WGQGTQVTVSS

SEQ ID NO: 36 - protein, 110 aa, synthetic construct
ELVLTQPTSVSGSLGQRVTISCTGSSSNIGRGYVGWYQQLPGTGPRTLIYDSSSRPSGVPDRFSGSRSGSTATLTISGLQAEDEADYYCSAYDNSLSGTVFGGGTHLTVL

SEQ ID NO: 37 - protein, 25 aa, synthetic construct
ELVLTQPTSVSGSLGQRVTISCTGS

SEQ ID NO: 38 - protein, 10 aa, synthetic construct
SSNIGRGYVG

SEQ ID NO: 39 - protein, 15 aa, synthetic construct
WYQQLPGTGPRTLIY

SEQ ID NO: 40 - protein, 4 aa, synthetic construct
DSSS

SEQ ID NO: 41 - protein, 35 aa, synthetic construct; identical to SEQ ID NO: 25

SEQ ID NO: 42 - protein, 9 aa, synthetic construct
SAYDNSLSG

SEQ ID NO: 43 - protein, 12 aa, synthetic construct
TVFGGGTHLTVL